(19) United States
(12) Patent Application Publication
(10) Pub. No.: US 2024/0066270 A1
(43) Pub. Date: Feb. 29, 2024

(54) Integrated Catheter System with Multiple Extension Sets

(71) Applicant: Becton, Dickinson and Company, Franklin Lakes, NJ (US)

(72) Inventors: Dorien Gemma Butter, Millcreek, UT (US); Jason Andrew Wine, Brea, CA (US); Jonathan Karl Burkholz, Salt Lake City, UT (US); Serena Robinson Agrawal, Salt Lake City, UT (US)

(21) Appl. No.: 18/238,588

(22) Filed: Aug. 28, 2023

Related U.S. Application Data
(60) Provisional application No. 63/401,930, filed on Aug. 29, 2022.

(51) Int. Cl. A61M 25/06; A61M 25/00; A61M 25/02; A61M 39/10

(52) U.S. Cl. CPC A61M 25/0606; A61M 25/0097; A61M 25/02; A61M 39/10; A61M 2025/0253

(57) ABSTRACT

An integrated catheter system includes a catheter adapter comprising a catheter, a body receiving the catheter, and a side port in fluid communication with the catheter, a first line in fluid communication with the catheter, a second line in fluid communication with the catheter, a first connector coupled to the first line, and a second connector coupled to the second line.

### CROSS-REFERENCE TO RELATED APPLICATION

The present application claims priority to U.S. Provisional Application Ser. No. 63/401,930 entitled “Integrated Catheter System with Multiple Extension Sets” filed Aug. 29, 2022, the entire disclosure of which is hereby incorporated by reference in its entirety.

### BACKGROUND OF THE INVENTION

### Field of the Invention

The present disclosure relates to an integrated catheter system with multiple extension set.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient. A vascular access device (VAD) may access peripheral vasculature of a patient. A VAD may be indwelling for short term (days), moderate term (weeks), or long term (months to years). VADs may be used for infusion therapy and/or for blood withdrawal.

A common type of VAD is an over-the-needle peripheral intravenous catheter (PIVC). Currently, there may be several limitations to the use of a PIVC for fluid infusion or blood draw. Among these limitations is the manipulation of the PIVC, for example, to manually connect various instruments thereto to enable blood draw; however, doing so in an uncontrolled way can increase incidence of dislodgement of the VAD from the vasculature, thus shortening the life of the catheter indwell. Accordingly, a need exists in the art for improved devices, systems, and methods of manipulating an indwelling catheter to free the catheter tip and allow for blood collection and fluid delivery therefrom or therethrough.

### SUMMARY OF THE INVENTION

In one aspect or embodiment, an integrated catheter system includes a catheter adapter having a catheter, a body receiving the catheter, and a side port in fluid communication with the catheter, a first line in fluid communication with the catheter, a second line in fluid communication with the catheter, a first connector coupled to the first line, and a second connector coupled to the second line.

The first connector and the second connector may each include a needle-free connector. The first line may include a near patient access port in fluid communication with the side port of the catheter adapter, with the near patient access port including a connector portion configured to be coupled to a peripheral probe device. The near patient access port further may include a secondary port, where the connector portion of the near patient access port is a needle-free connector. The system may further include a third line in fluid communication with the secondary port of the near patient access port, with the third line including an access port.

The catheter adapter may include a secondary side port, with the first line coupled to the side port of the catheter adapter, and the second line coupled to the secondary side port of the catheter adapter. The secondary side port may be positioned on an opposite side of the body of the catheter adapter relative to the side port.

The system may further include a stabilization platform spaced from the body of the catheter adapter and positioned along the first line or the second line. The stabilization platform may include an attachment member configured to secure the stabilization platform to a skin surface of a patient. The attachment member may include an adhesive pad. The system may further include a clamp positioned along at least one of the first line and the second line. The stabilization platform may include a pair of stabilizing wings. The catheter adapter may include a pair of catheter adapter stabilizing wings. The stabilization platform may be moveable along the first line or the second line.

At least one of the first connector and the second connector may include a force controlled release connector, where at least a portion of the force controlled release connector is configured to separate from the proximal access port when a predetermined separation force is applied to the force controlled release connector. The force controlled release connector may include a disconnect connector and a release feature, with the release feature engaged with the disconnect connector and configured to be disengaged from the disconnect connector when a predetermined separation force is applied to the disconnect connector. The disconnect connector may include a valve member. The disconnect connector may include a primary line extending from the disconnect connector and a secondary line extending from the disconnect connector, where the primary line includes a first port and the secondary line includes a second port. The first line may extend in a distal direction and the second line may extend in a proximal direction.

The system may include a stabilization platform spaced from the body of the catheter adapter and distal of the catheter, with the stabilization platform positioned along the first line. The catheter adapter may include a pair of catheter adapter stabilizing wings, where a stabilizer arm extends between the stabilization platform and the catheter adapter stabilizing wings.

### BRIEF DESCRIPTION OF THE DRAWINGS

FIG. 1 is a top view of an integrated catheter system according to one aspect or embodiment of the present application;

FIG. 2 is a perspective view of an integrated catheter system according to a further aspect or embodiment of the present application;

FIG. 3 is a perspective view of an integrated catheter system according to a further aspect or embodiment of the present application;

FIG. 4 is a perspective view of an integrated catheter system according to a further aspect or embodiment of the present application;

FIG. 5 is a top view of an integrated catheter system according to a further aspect or embodiment of the present application;

FIG. 6 is a top view of an integrated catheter system according to a further aspect or embodiment of the present application;

FIG. 7 is a top view of a force controlled release connection according to one aspect or embodiment of the present application;

FIG. 8 is a cross-sectional view of a force controlled release connection according to a further aspect or embodiment of the present application;

FIG. 9 is a perspective view of an integrated catheter system according to a further aspect or embodiment of the present application;

FIG. 10 is a top view of an integrated catheter system according to a further aspect or embodiment of the present application;

FIG. 11 is a top view of an integrated catheter system according to a further aspect or embodiment of the present application;

FIG. 12 is a top view of an integrated catheter system according to a further aspect or embodiment of the present application;

FIG. 13 is a view of an integrated catheter system according to a further aspect or embodiment of the present application;

FIG. 14 is a view of an integrated catheter system according to a further aspect or embodiment of the present application;

FIG. 15 is a view of an integrated catheter system according to a further aspect or embodiment of the present application;

FIG. 16 is a view of an integrated catheter system according to a further aspect or embodiment of the present application; and

FIG. 17 is a view of an integrated catheter system according to a further aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

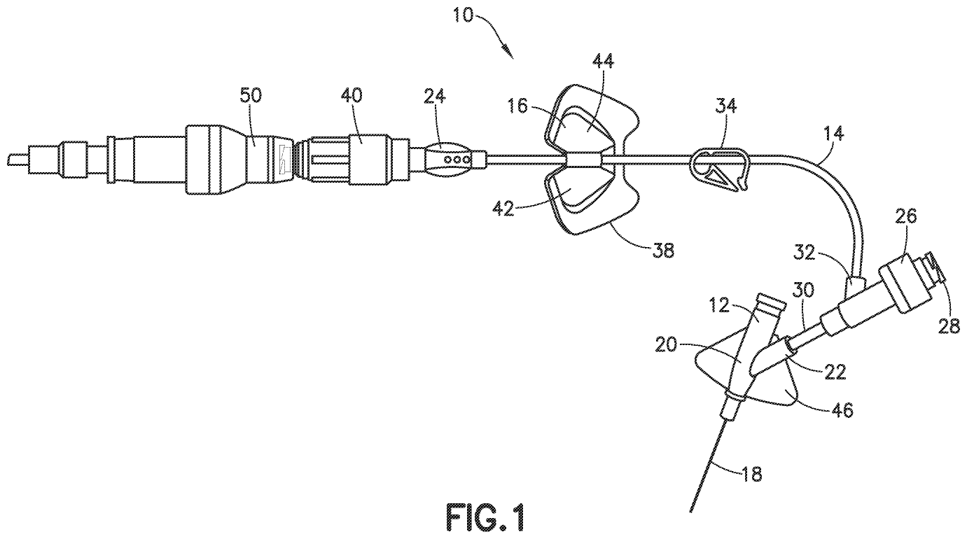

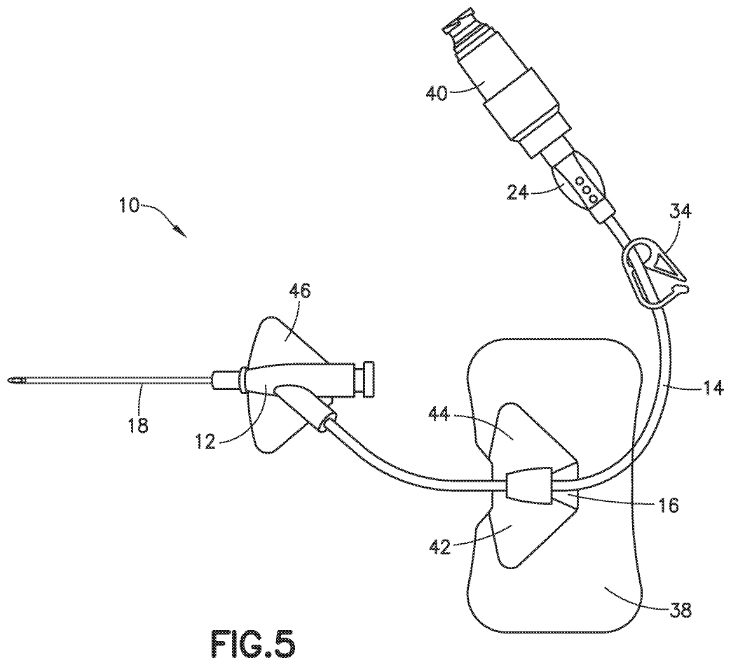

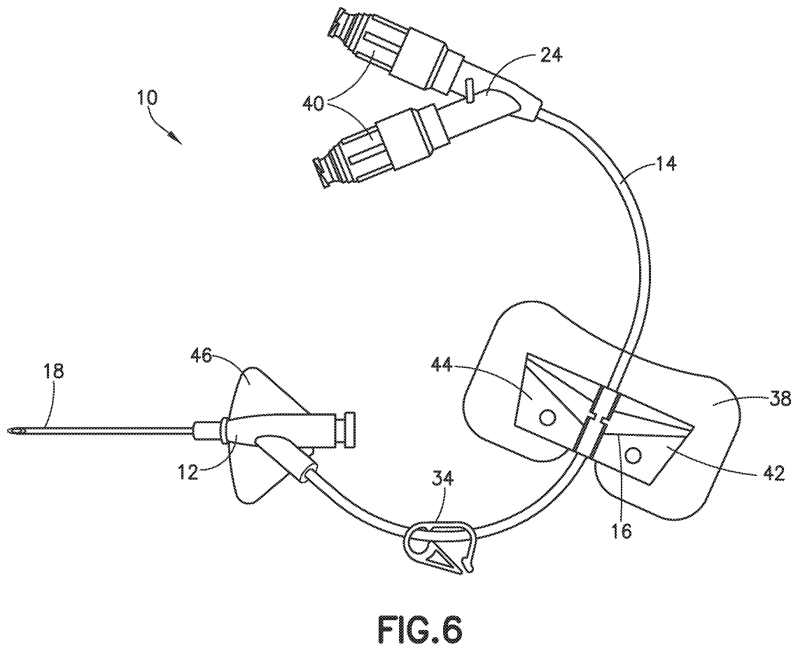

Referring to FIGS. 1-6, according to one aspect or embodiment, an integrated catheter system 10 includes a catheter adapter 12, an integrated extension set 14, and a stabilization platform 16. The catheter adapter 12 includes a catheter 18, a body 20 receiving the catheter 18, and a side port 22 in fluid communication with the catheter 18. The integrated extension set 14 includes a proximal access port 24 coupled to a proximal end portion of the integrated extension set 14, with the integrated extension set 14 in fluid communication with the side port 22 of the catheter adapter 12. The stabilization platform 16 is spaced from the body 20 of the catheter adapter 12 and positioned along the integrated extension set 14. The stabilization platform 16 is configured to provide a primary securement point to a skin surface of a patient and minimize catheter dislodgement, infiltration, or catheter movement that may result in a tip of the catheter 18 from being pulled out of a vein or artery. The stabilization platform 16 is configured to isolate external applied forces from the catheter adapter 12 and insertion site to limit movement of the catheter 18 and/or dislodgement of the catheter 18 due to forces being applied to an intravenous line or extension sets. The system 10 is configured to provide sufficient compliance, flex, or slack between the stabilization platform 16 and the catheter adapter 12 to allow some displacement of the stabilization platform 16 relative to the catheter 18 due to movement of the skin around the stabilization platform 16 without applying excessive force and displacement of the catheter adapter 12 itself. In some aspects or embodiments, the system 10 provides a high pressure rated catheter with blood draw and instrument delivery access and high pressure injection capabilities.

Referring to FIG. 1, for example, the system 10 may include a near patient access port 26 in fluid communication with the side port 22 of the catheter adapter 12, with the near patient access port 26 including a connector portion 28 configured to be coupled to a peripheral probe device. The near patient access port 26 is configured to provide catheter access to peripheral devices such as, e.g., a blood draw device (e.g., PIVO™ from Becton, Dickinson and Company), or a vascular access probe (VAP) for in-vein digital measurement of patient data such as temperature, pH, lactate, and/or other blood-based measurements. In some embodiments, the near patient access port 26 is coupled to the side port 22 of the catheter adapter 12 via a length of intermediate tubing 30. However, in other embodiments, the near patient access port 26 may be coupled directly to the side port 22, or connected via another intermediate member.

The near patient access port 26 includes the connector portion 28, which in some embodiments, is configured to be compatible with peripheral devices such as blood draw devices and/or vascular access probes. In some embodiments, the connector portion 28 is configured as a needle-free connector (NFC) configured to receive a blunt introducer of a blood draw device. More specifically, the connector portion 28 may be configured as a split-septum NFC with direct probe access such as, e.g., Q-Syte™ or SmartSite™ NFCs from Becton, Dickinson and Co., or any other appropriate split-septum NFC. Alternatively, in other embodiments, the connector portion 28 may be formed of a non-split-septum-type NFC. Furthermore, in some embodiments, the near patient access port 26 may include anti-microbial and/or flush-promoting features. For example, the near patient access port 26 may include one or more of an offset tubing port vortex-creating feature, a proximal flow-diverting feature, anti-microbial NFC lubricant, anti-microbial eluting surface coating(s) or insert(s), etc. With the near patient access port 26 fluidly coupled to the catheter adapter 12 via the side port 22, the system 10 provides for probe (or tube) access from a peripheral probe device through the indwelling catheter 18. The near patient access port 26 further includes a secondary port 32 positioned near a distal end thereof. In some embodiments, the secondary port 32 is coupled to the integrated extension set 14. A clamp 34 may be provided on the integrated extension set 14, with the clamp 34 configured to selectively restrict flow through the integrated extension set 14.

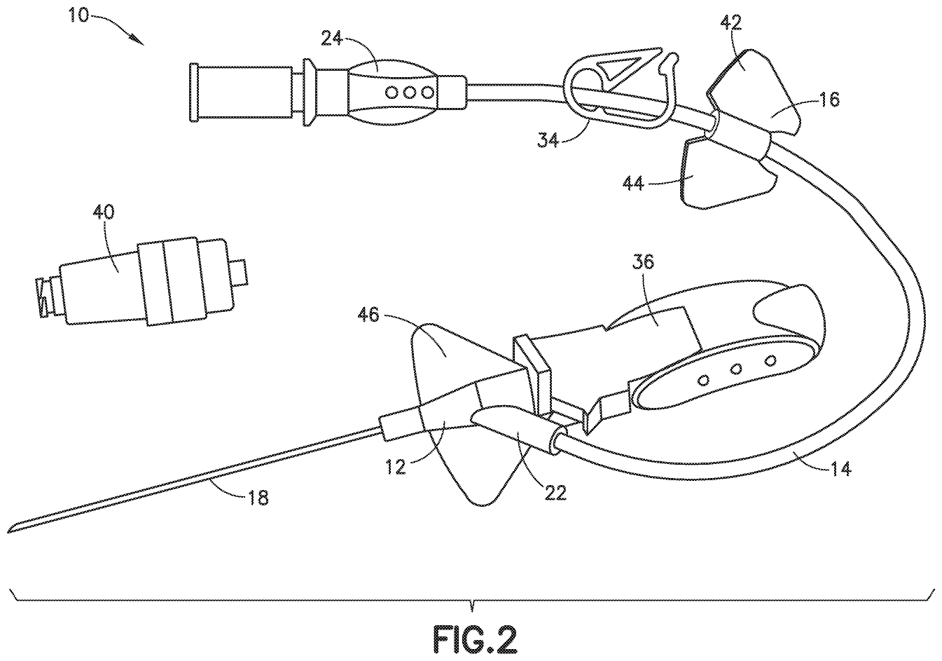

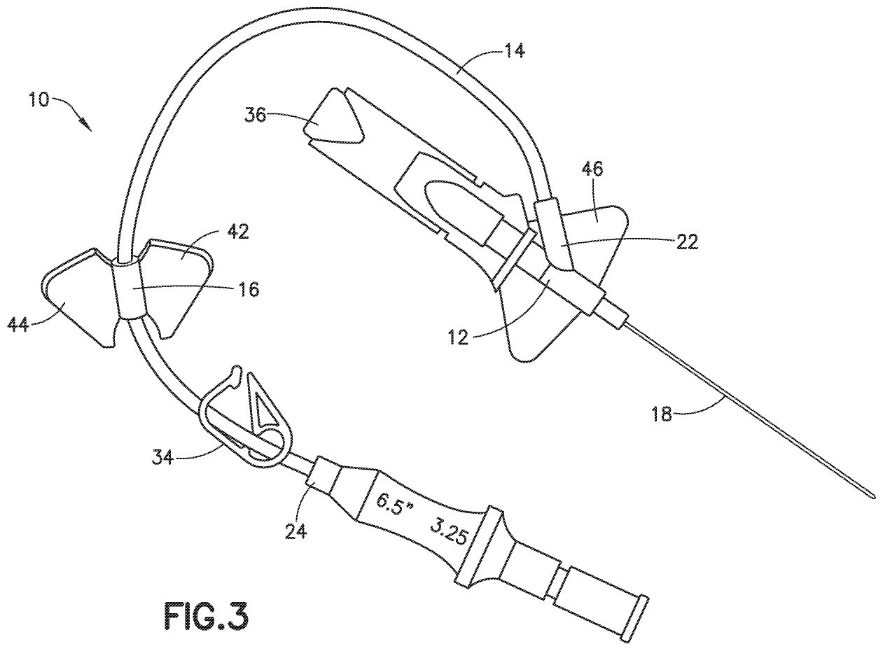

Referring to FIGS. 2 and 3, in some aspects or embodiments, the integrated extension set 14 extends directly from the side port 22 of the catheter, with the system 10 not including the near patient access port 26. The system 10 may incorporate the BD Nexiva™ closed peripheral IV catheter system (FIG. 2) or the BD Nexiva™ Diffusics™ closed IV catheter system (FIG. 3). The catheter 18 of any of the aspects or embodiments described herein may be inserted according to any suitable arrangement or method. The system 10 may include a needle insertion device 36 to facilitate the insertion of the catheter 18.

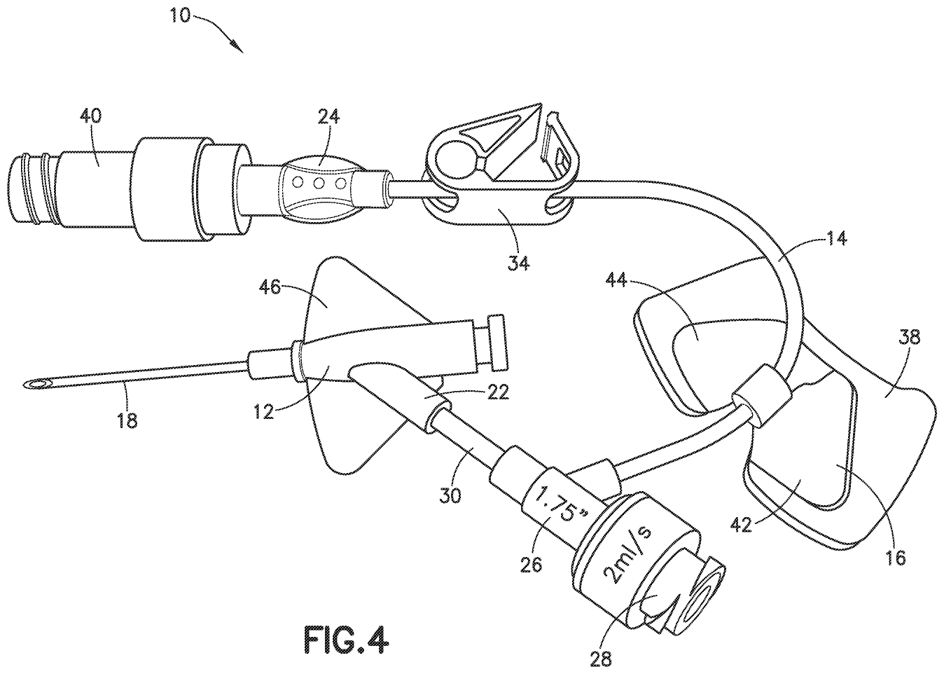

Referring to FIGS. 4-6, in one aspect or embodiment, the stabilization platform 16 includes an attachment member 38 configured to secure the stabilization platform 16 to a skin surface of a patient. The attachment member 38 may include an adhesive pad. The attachment member 38 may be securement tape, an external stabilization device (ESD), dressing, and/or an anchoring platform adhesive with paper backing and skin adhesive. In one aspect or embodiment, the stabilization platform 16 is shaped to fit within the StatLock™ ESD product from Becton, Dickinson and Co.

Referring again to FIGS. 1-6, in some aspects or embodiments, the stabilization platform 16 is positioned distal to the proximal access port 24 and proximal to the clamp 34. In some aspects or embodiments, the stabilization platform 16 is positioned distal to the clamp 34. The stabilization platform 16 may also be integrated or coupled to the proximal access port 24 or the near patient access port 26. The proximal access port 24 may include a needle-free connector 40 integrated into the proximal access port 24 or separately connected to the proximal access port 24. The stabilization platform 16 includes a pair of stabilizing wings 42, 44, although other suitable arrangements may be utilized. The stabilization platform 16 may be fixed, removable, or moveable along the integrated extension set. The stabilization platform 16 may be oval, triangular, round, square, rectangular, trapezoidal, or other suitable shape. The stabilization platform 16 may be flexible, soft, semi-rigid, or rigid and may include features or a living hinge to promote flexibility for patient comfort. The stabilization platform 16 may also include one or more features to improve attachment to the attachment member 38, such as protrusions, surface roughness, pockets, etc. The stabilization platform 16 may include one or more colors or visual marking to provide catheter gauge, length, type, or procedure related information. In some aspects or embodiments, the catheter adapter 12 includes a pair of catheter adapter stabilizing wings 46.

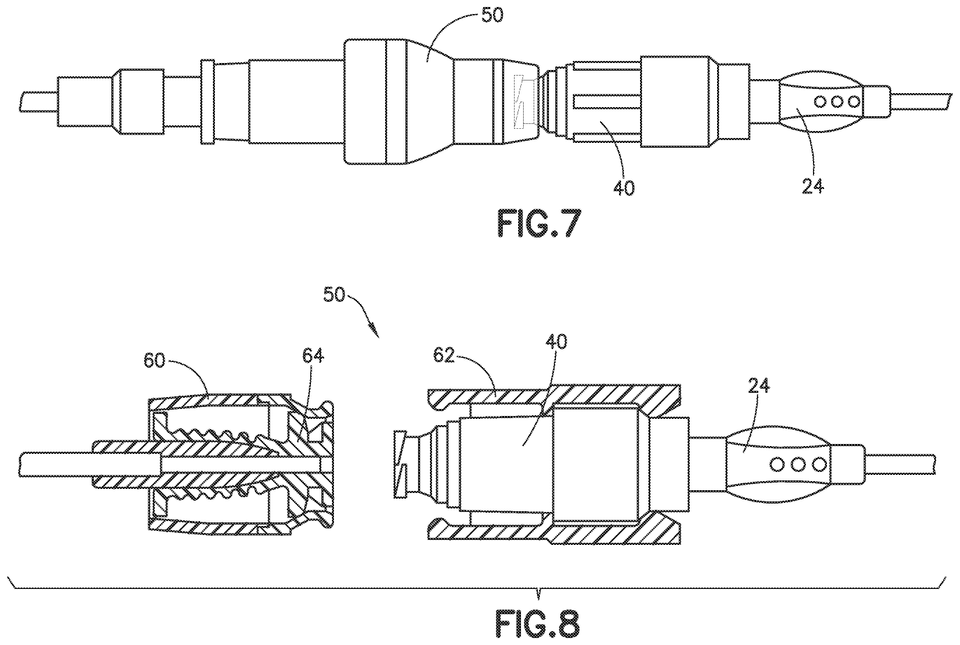

Referring to FIGS. 1 and 7-9, in some aspects or embodiments, the system 10 further includes a force controlled release connector 50 coupled to the proximal access port 24, or a needle-free connector 40 that is connected to access port 24. At least a portion of the force controlled release connector 50 is configured to separate from the proximal access port 24 when a predetermined separation force is applied to the force controlled release connector 50. At least a portion of the force controlled release connector 50 is configured to detach from the integrated extension set 14 to prevent excessive forces from being transmitted to the catheter adapter 12 via the integrated extension set 14. For example, a line from an IV bag may be connected to the integrated extension set 14 via the force controlled release connector 50 such that excessive movement of the IV bag and line will merely detach the connection between the integrated extension set 14 and the force controlled connector 50 rather than disrupting the placement of the catheter 18.

Referring to FIG. 8, the force controlled release connector 50 includes a disconnect connector 60 and a release feature 62 coupled to the needle-free connector 40 of the proximal access port 24. The release feature 62 is engaged with the disconnect connector 60 and configured to be disengaged from the disconnect connector 60 when a predetermined separation force is applied to the disconnect connector 60 or the needle-free connector 40 of the proximal access port 24. The disconnect connector 60 includes a valve member 64, which is configured to seal the disconnect connector 60 when separated from the needle-free connector 40. In one aspect or embodiment, the release feature 62 is configured to be biased radially outward when the predetermined separate force is reached. The release feature 62 and the disconnect connector 60 may form a camming arrangement where an axial force causes the release feature 62 to move radially outward, thereby releasing the disconnect connector 60 from the release feature 62. The release feature 62 may be formed integrally with the needle-free connector 40 or may be a separate component attached or coupled to the needle-free-connector 40 or the proximal access port 24.

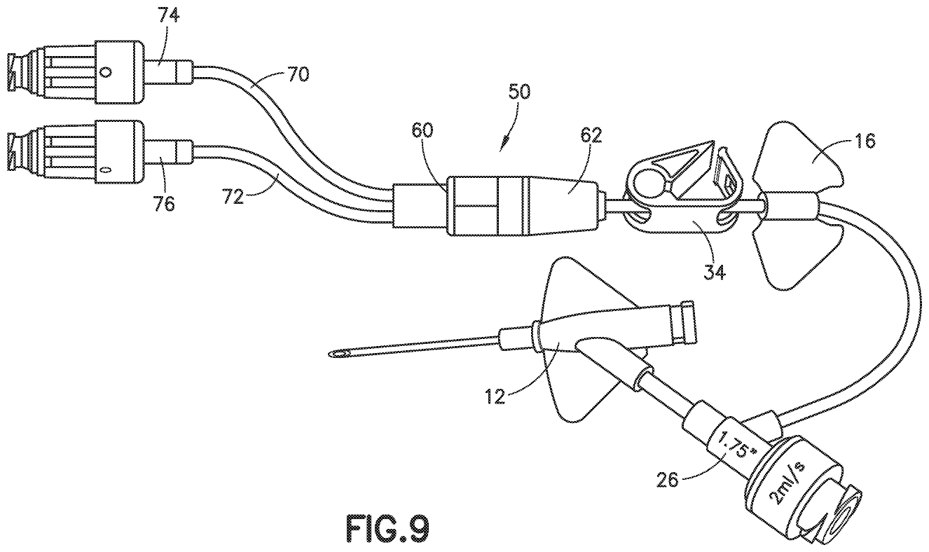

Referring to FIG. 9, in some aspects or embodiments, the disconnect connector 60 includes a first line 70 extending from the disconnect connector and a second line 72 extending from the disconnect connector 60. The first line 70 includes a first port 74 and the second line includes a second port 76. The first and second ports 74, 76 may each include a needle-free connector.

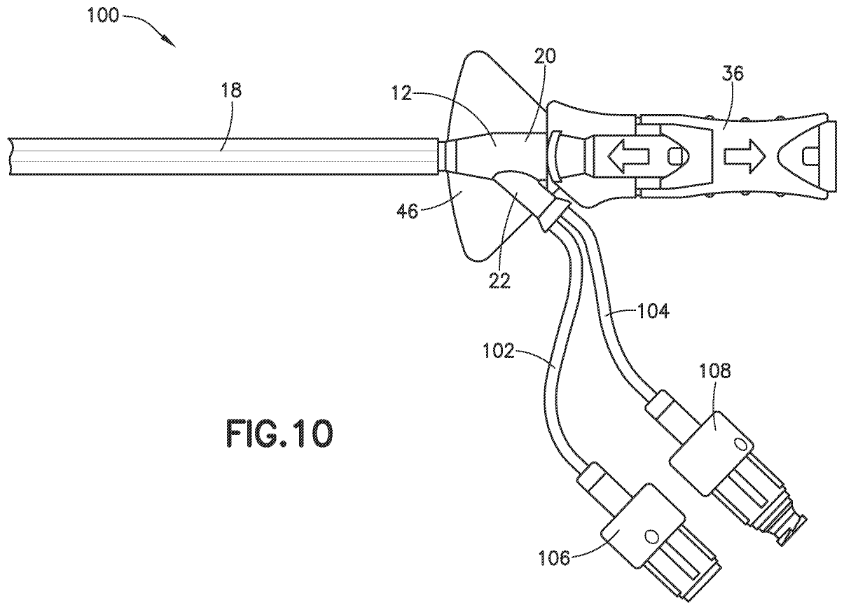

Referring to FIGS. 10-17, in one aspect or embodiment, an integrated catheter system 100 includes the catheter adapter 12, a first line 102 in fluid communication with the catheter 18 (which may include a needle cover, as shown in FIGS. 10-13), a second line 104 in fluid communication with the catheter 18, a first connector 106 coupled to the first line 102, and a second connector 108 coupled to the second line 104. As discussed in additional detail below, although two lines are shown in FIG. 10, for example, each line may include one or more additional lines or connectors. Including multiple extension sets allows for multiple applications and expands the capability of the system 100. One extension set or line may be shorter and/or stiffer to allow for uses with probes or blood collection devices and other extension sets or lines may be longer and more flexible to allow for traditional IV uses and applications. As shown in FIG. 10, in one aspect or embodiment, the first and second connectors 106, 108 are each a needle-free connector, with the first and second lines 102, 104 extending from the side port 22 of the catheter adapter 12. The first and second lines 102, 104 may be medical tubing.

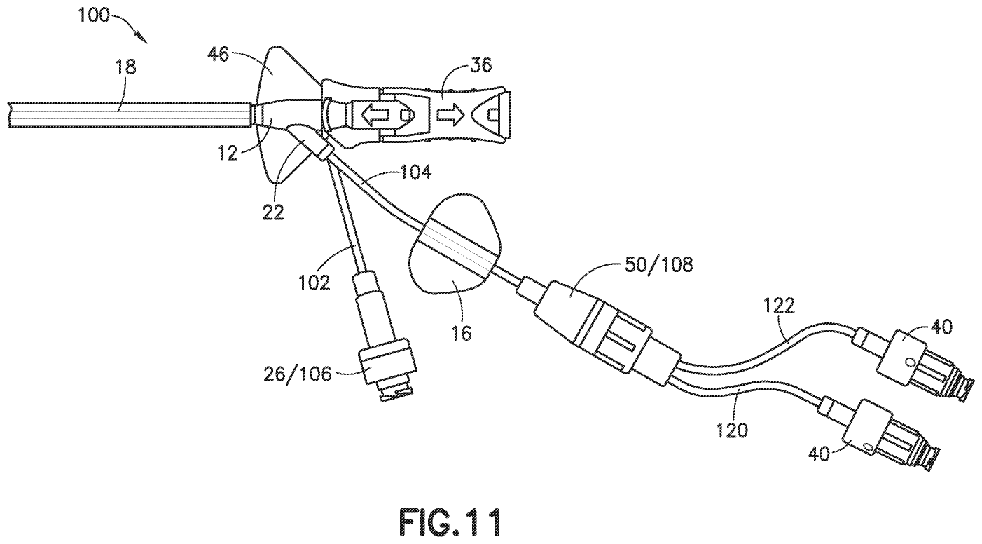

Referring to FIG. 11, in one aspect or embodiment, the system 100 includes the stabilization platform 16 according to any of the aspects or embodiments discussed above. The first connector 106 of the first line 102 includes the near patient access port 26 as detailed above and the second connector 108 of the second line 104 includes the force controlled release connector 50 of any of the aspects or embodiments discussed above. The stabilization platform 16 may be provided on either the first line 102 or the second line 104, although shown positioned along the second line 104 in FIG. 11. The force controlled release connector 50 may include a primary line 110 and a secondary line 112 extending from the disconnect connector 60.

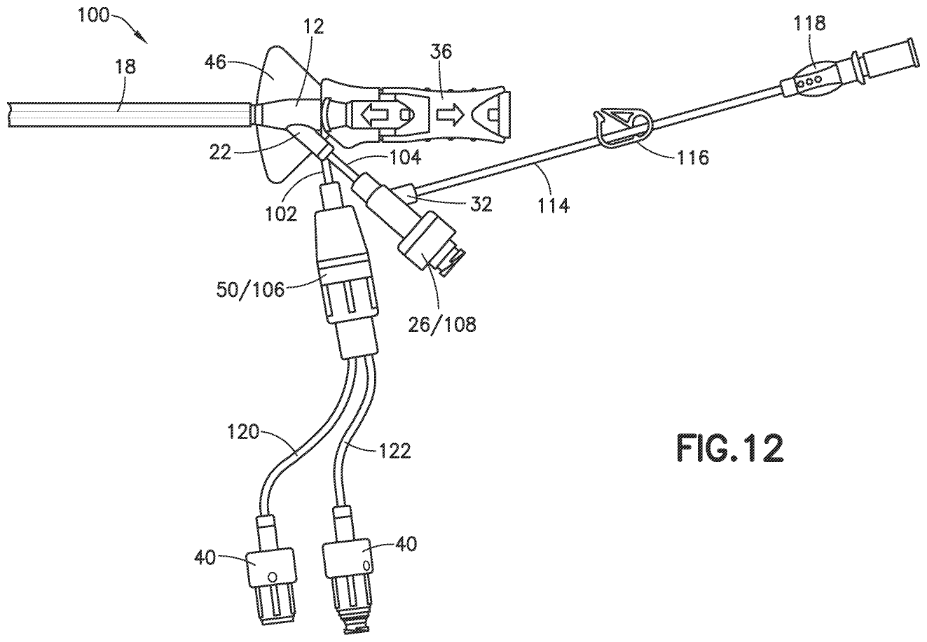

Referring to FIG. 12, in one aspect or embodiment, the first connector 106 of the first line 102 includes the force controlled release connector 50 of any of the aspects or embodiments discussed above and the second connector 108 of the second line 104 includes the near patient access port 26 as detailed above. A third line 114 extends from the secondary port 32 of the near patient access port 26. A clamp 116 is positioned along the third line 114. The third line 114 includes an access port 118, which may include a luer connector, needle-free connector, and/or a vent plug. The force controlled release connector 50 may include a primary line 120 and a secondary line 122 extending from the disconnect connector 60.

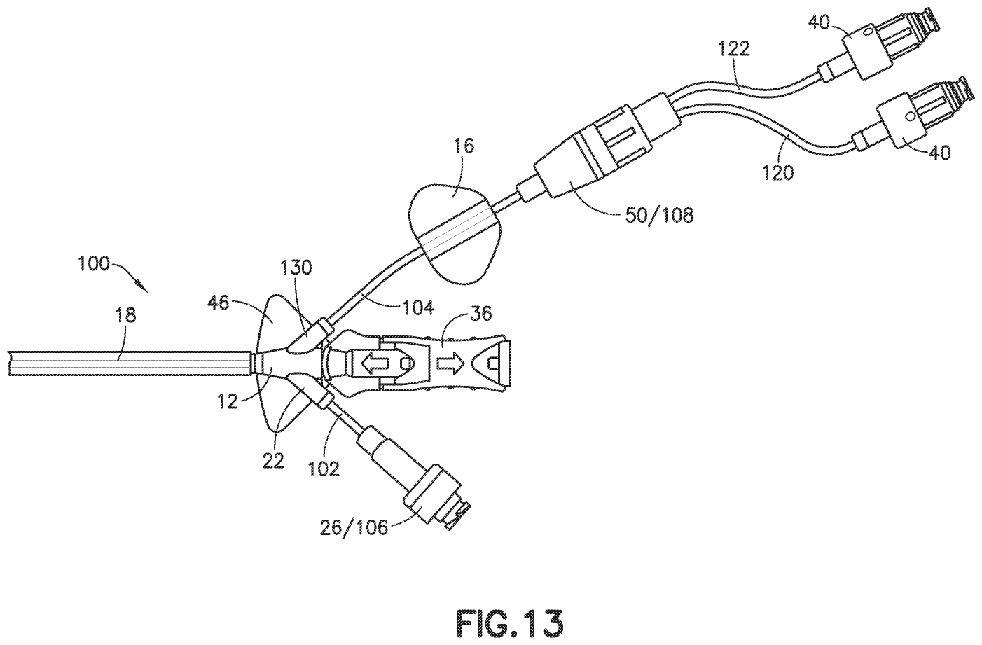

Referring to FIG. 13, in one aspect or embodiment, the catheter adapter 12 includes a secondary side port 130, with the first line 102 coupled to the side port 22 of the catheter adapter 12, and the second line 104 coupled to the secondary side port 130 of the catheter adapter 12. The first connector 106 of the first line 102 includes the near patient access port 26 as detailed above and the second connector 108 of the second line 104 includes the force controlled release connector 50 of any of the aspects or embodiments discussed above. The force controlled release connector 50 may include the primary line 110 and the secondary line 112 extending from the disconnect connector 60.

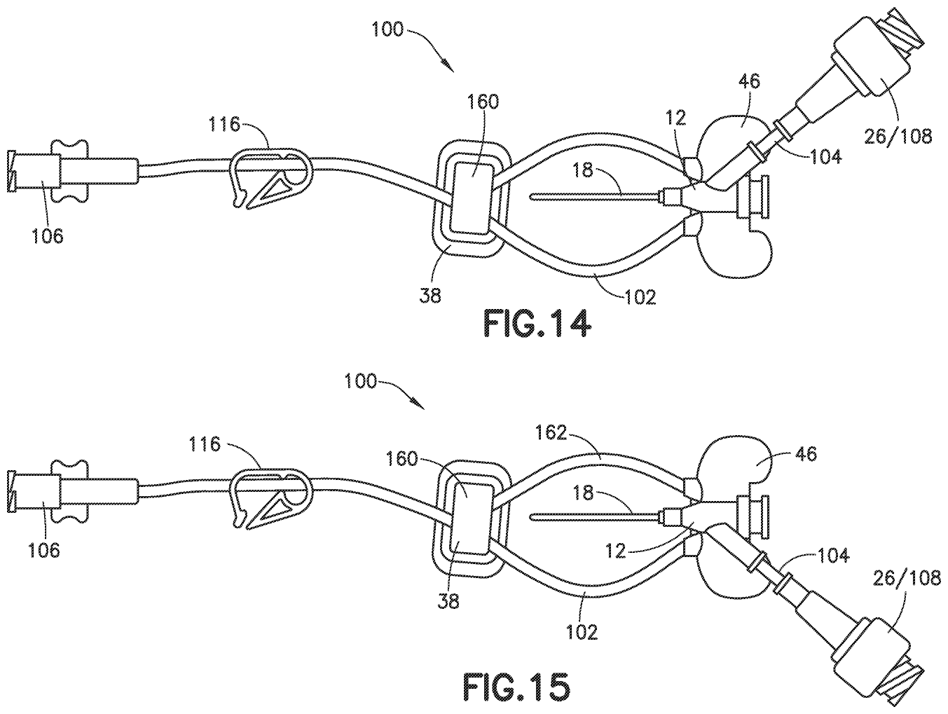

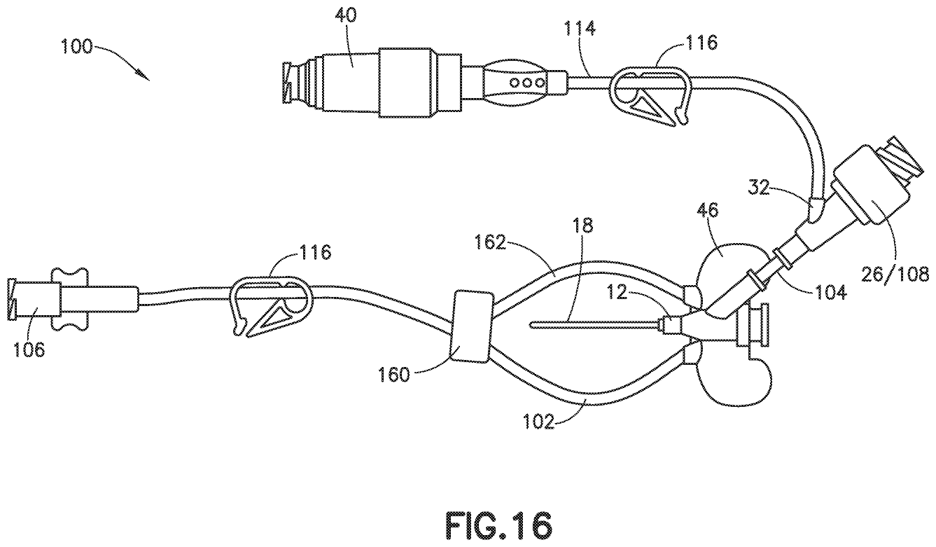

Referring to FIGS. 14-16, in one aspect or embodiment, the system 100 includes a stabilization platform 160 spaced from the body 20 of the catheter adapter 12 and distal of the catheter 18, with the stabilization platform 160 positioned along the first line 102. The stabilization platform 160 is similar to and functions similarly to the stabilization platform 16 discussed above. The stabilization platform 160 includes a stabilizer arm 162 that extends between the stabilization platform 160 and the catheter adapter stabilizing wings 46 of the catheter adapter 12. The first line 102 extends in a distal direction and the second line 104 extends in a proximal direction. The second line 104 extends in a proximal direction, with the second connector 108 including the near patient access port 26, although other suitable connectors and configurations may be utilized. The second line 104 may be positioned on the same side of the catheter adapter 12 as the first line 102 (FIG. 15) or the second line 104 may be positioned on an opposite side of the catheter adapter 12 relative to the first line 102 (FIG. 14). The second connector 108 may also include the near patient access port 26 (as shown in FIG. 13), with the third line 114 extending from the secondary port 32 (as shown in FIG. 16).

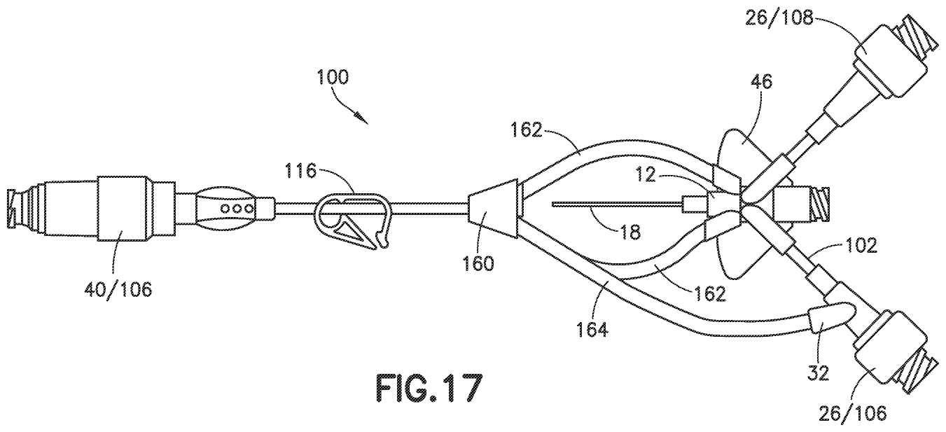

Referring to FIG. 17, in one aspect or embodiment, the stabilization platform 160 includes two stabilizer arms 162 that extend between the stabilization platform 160 and the catheter adapter stabilizing wings 46 of the catheter adapter 12. The first line 102 extends from the side port 22 and the second line 104 extends from the secondary side port 130 of the catheter adapter 12, as shown in FIG. 13. The first connector 106 of the first line 102 includes the near patient access port 26, with an extension line 164 extending in a distal direction from the secondary port 32 of the near patient access port 26. The extension line 164 extends through the stabilization platform 160. By arranging one or more stabilizer arms 162 and/or line in the manner shown in the accompanying figures, a force can be applied to the stabilizer platform 160 and, consequently, the catheter 18, such that the catheter 18 is pulled further into the vasculature, rather than inadvertently being pulled out of the patient's vasculature. In addition, properly securing the anchor platform 160, and providing for curvature in the arms 162 and/or distally directed extension rube 162, 114, can allow for externally applied forces to be isolated from the catheter adapter thereby minimizing movement of the catheter adapter and catheter due to the applied force(s).

While several embodiments of integrated catheter systems were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is:

1. An integrated catheter system comprising:
a catheter adapter comprising a catheter, a body receiving the catheter, and a side port in fluid communication with the catheter;
a first line in fluid communication with the catheter;
a second line in fluid communication with the catheter;
a first connector coupled to the first line; and
a second connector coupled to the second line.

2. The system of claim 1, wherein the first connector and the second connector each comprise a needle-free connector.

3. The system of claim 1, wherein the first line comprises a near patient access port in fluid communication with the side port of the catheter adapter, the near patient access port comprising a connector portion configured to be coupled to a peripheral probe device.

4. The system of claim 3, wherein the near patient access port further comprises a secondary port, and wherein the connector portion of the near patient access port is a needle-free connector.

5. The system of claim 4, further comprising a third line in fluid communication with the secondary port of the near patient access port, the third line comprising an access port.

6. The system of claim 1, wherein the catheter adapter comprises a secondary side port, the first line coupled to the side port of the catheter adapter, and the second line coupled to the secondary side port of the catheter adapter.

7. The system of claim 6, wherein the secondary side port is positioned on an opposite side of the body of the catheter adapter relative to the side port.

8. The system of claim 1, further comprising a stabilization platform spaced from the body of the catheter adapter and positioned along the first line or the second line.

9. The system of claim 8, wherein the stabilization platform comprises an attachment member configured to secure the stabilization platform to a skin surface of a patient.

10. The system of claim 9, wherein the attachment member comprises an adhesive pad.

11. The system of claim 1, further comprising a clamp positioned along at least one of the first line and the second line.

12. The system of claim 8, wherein the stabilization platform comprises a pair of stabilizing wings.

13. The system of claim 1, wherein the catheter adapter comprises a pair of catheter adapter stabilizing wings.

14. The system of claim 8, wherein the stabilization platform is moveable along the first line or the second line.

15. The system of claim 1, wherein at least one of the first connector and the second connector comprises a force controlled release connector, and wherein at least a portion of the force controlled release connector is configured to separate from the proximal access port when a predetermined separation force is applied to the force controlled release connector.

16. The system of claim 15, wherein the force controlled release connector comprises a disconnect connector and a release feature, the release feature is engaged with the disconnect connector and configured to be disengaged from the disconnect connector when a predetermined separation force is applied to the disconnect connector.

17. The system of claim 16, wherein the disconnect connector comprises a valve member.

18. The system of claim 16, wherein the disconnect connector comprises a primary line extending from the disconnect connector and a secondary line extending from the disconnect connector, and wherein the primary line comprises a first port and the secondary line comprises a second port.

19. The system of claim 1, wherein the first line extends in a distal direction and the second line extends in a proximal direction and further comprising a stabilization platform spaced from the body of the catheter adapter and distal of the catheter, the stabilization platform positioned along the first line.

20. The system of claim 19, wherein the catheter adapter comprises a pair of catheter adapter stabilizing wings, and wherein a stabilizer arm extends between the stabilization platform and the catheter adapter stabilizing wings.
